(19) United States
(12) Patent Application Publication
(10) Pub. No.: US 2018/0110423 A1
(43) Pub. Date: Apr. 26, 2018

(54) OPTICAL LASER SPECKLE SENSOR FOR MEASURING A BLOOD PERFUSION PARAMETER

(71) Applicant: KONINKLIJKE PHILIPS N.V., EINDHOVEN (NL)

(72) Inventors: Cristian Nicolae Presura, Veldhoven (NL); Lisa Opstal, Eindhoven (NL); Francesco Sartor, Eindhoven (NL); Calina Ciuhu, Eindhoven (NL)

(21) Appl. No.: 15/565,969

(22) PCT Filed: Apr. 15, 2016
(86) PCT No.: PCT/EP2016/058360

§ 371 Date: Oct. 12, 2017

(30) Foreign Application Priority Data
Apr. 15, 2015 (EP) 15163714.7

(51) Int. Cl. A61B 5/026; A61B 5/00; A61B 5/0205

(52) U.S. Cl. CPC A61B 5/0261; A61B 5/746; A61B 5/02055; A61B 5/02427; A61B 5/6843; A61B 2562/0238; A61B 5/7214

(57) ABSTRACT

The invention relates to an optical sensor device (1) for determining a blood perfusion parameter of a user. A light source (2) provides coherent light for scattering in a tissue sample (11) and a light detection unit (3) receives scattered coherent light in a re-emission geometry, the light detection unit (3) comprising plural light detection elements (32a; 32b) for capturing light intensity values in increasing distances from the light source (2) in accordance with different tissue depths. An evaluation unit (10) determines contrast values based on the captured light intensities, determines one motion-corrected value of the blood perfusion parameter based on the contrast values associated with the different tissue depths. Moreover, the invention relates to a method determining at least one blood perfusion parameter using the sensor device (1).

### FIELD OF THE INVENTION

The invention relates to the measurement of a blood perfusion parameter using an optical sensor. More specifically, the invention is related to an optical sensor device for determining at least one blood perfusion parameter, which comprises a light source for providing coherent light for scattering in a tissue sample of a user and a light detection unit for receiving at least part of the scattered coherent light and for capturing at least one light intensity value in accordance with a speckle pattern formed by scattered coherent light. Moreover, the invention is related to a method for determining at least one blood perfusion parameter of a user.

### BACKGROUND OF THE INVENTION

It is known that the perfusion of blood in human tissue is correlated to the health condition of an individual. One parameter related to blood perfusion is the heart frequency of the individual, where extreme values of the heart frequency can be indicative of detrimental health conditions of the individual. Therefore, the monitoring of the heart frequency can provide indications of detrimental health conditions of the individual. Further, the monitoring of the heart frequency allows for assessing the performance of an individual during sporting activities or the like. Moreover, the blood velocity is a further blood perfusion parameter that is correlated to the physiological condition of an individual. In particular, an irregular blood velocity can be indicative of cardiovascular diseases, such as hypertension and atherosclerosis, coronary artery disease, chronic heart failure, peripheral vascular disease, stroke, diabetes, chronic kidney failure, and infectious diseases.

One option for determining and monitoring such blood perfusion parameters involves laser speckle imaging. Here, a tissue sample is illuminated using coherent laser light which is scattered by red blood cells within the tissue. The scattered light produces an interference pattern which is usually also referred to as speckle pattern. The movement of the blood cells causes changes of the speckle pattern which particularly result in a blurring of a measured speckle pattern (i.e. of an image of the speckle pattern). Therefore, the amount of blurring which can be parameterized by the contrast of the speckle pattern is correlated to movement of the blood cells. Consequently, blood perfusion parameters can be estimated on the basis of the contrast of measured speckle patterns.

In this respect, US 2013/0204112 A1 discloses a method and an apparatus for monitoring blood perfusion using laser speckle imaging, which includes a coherent light source and a detector for measuring light transmitting a tissue sample. On the basis of variations in the transmitted light, the apparatus determines speckle contrast values. These contrast values are used for computing a metric of blood perfusion.

Since the light source and the detector are positioned relative to each other in transmission geometry in this apparatus, the apparatus has to be affixed to a part of the user's body which has a thickness sufficiently small for being transilluminated by the laser light. Therefore, the apparatus can only be affixed to a finger, toe, nostril or earlobe of the user. This limits the range of use of the apparatus. Moreover, it is usually uncomfortable for the users to have the apparatus attached to one of the aforementioned parts of their bodies, particularly when they use the apparatus during a longer period of time.

US 2012/0184831 A1 discloses a device for monitoring hemodynamics. The device directs light toward an area of a body and detects the resulting speckle pattern. The device is operated in reflectance geometry and includes a sensor patch in the form of a housing which can be mounted on the skin. On the basis of fluctuations of the speckle pattern, the device can determine the blood velocity and the heart rate. Further, US 2012/0184831 A1 discloses that the device can comprise more than one laser light source and/or light detector, and the separation distances can be different to enable simultaneous measurements with respect multiple tissue depths

### SUMMARY OF THE INVENTION

It is an object of the invention to allow for a monitoring of a blood perfusion parameter of a user in a flexible way and in a manner which is convenient for the user.

In a first aspect of the invention, an optical sensor device for determining at least one blood perfusion parameter of a user is suggested. The optical sensor device comprises a light source for providing coherent light for scattering in a tissue sample of the user and a light detection unit for receiving at least part of the scattered coherent light. The light source and the light detection unit are arranged relative to each other in a re-emission geometry. The light detection unit comprises plural light detection elements for capturing light intensity values in accordance with a speckle pattern formed by scattered coherent light, the light detection elements being arranged in increasing distances from the light source and being associated with different tissue depths. Further, the sensor device comprises a housing including the light source and the light detection unit, the housing comprising a contact surface which can be brought into contact with the tissue sample and which comprises a first opening through which light emitted by the light source can leave the housing and a second opening through which scattered light captured by the light detection unit can enter the housing. Moreover, the optical sensor devices comprises an evaluation unit configured to determine separate contrast values for the light detection elements based on the captured light intensities and to determine one motion-corrected value of the blood perfusion parameter based on the contrast values associated with the different tissue depths.

The suggested sensor device can be used more flexibly, since the light source and the light detection unit are arranged relative to each other in a re-emission geometry.

This does particularly mean that the light source and the light detection unit are arranged relative to each other such that the tissue sample is not positioned between the light source and the light detection unit. Thus, the sensor device can also be attached to thicker parts of the body, which can not be transilluminated by the light emitted by the light source. This does also allow for attaching the sensor device to a part of the user's body where the user can more comfortably carry the sensor device. In particular, the sensor device can be attached to the user's wrist so that the user can carry the sensor device like a wrist watch.

Moreover, relative movements of the tissue sample and the sensor device can be eliminated or minimized since the housing of the sensor device has a contact surface which comprises openings through which light emitted by the light source can leave the housing and through which scattered light can enter the housing and which can be brought into contact with the tissue sample. Such relative movements of the tissue sample and the sensor would also decrease the speckle contrast and, thus, would affect the determination of the blood perfusion parameter.

Further, the invention allows for assessing the velocity distribution of the blood within different layers of the tissue sample and to determine a motion-corrected value of the blood perfusion parameter. When the user moves, the tissue sample covered by the sensor device may partly move relative to the sensor device due to tensions in the tissue sample caused by motions of other parts of the body and or due to the motion of tendons or the like within the tissue sample. Particularly when the sensor device is worn at the user's wrist, the tissue sample may partly move relative to the sensor device due to movements of the user's hand and/or fingers. Such movement of the tissue sample lead to undesired motion artifacts in the determination of the blood perfusion parameter, since a reduction of the speckle contrast due to such movements may erroneously be treated as an indication of an increased blood perfusion.

In order to eliminate such motion artifacts, the evaluation unit is configured to determine one motion-corrected value of the blood perfusion parameter based on the contrast values of the blood perfusion parameter associated with the different tissue depths. Here, the fact is exploited that the aforementioned relative motions between the tissue sample and the sensor device affect different layers of the tissue sample in different tissue depths to a different extent. Therefore, it is possible to correct for such movements by calculating the blood perfusion parameter on the basis of the contrast values associated with the different tissue depths.

With respect to the re-emission geometry in which the light source and the light detection unit are arranged, one embodiment of the invention provides that the first and second openings are arranged relative to each other in such a way that a connection line between the first and second openings does not cross the tissue sample, when the contact surface is in contact with the tissue sample.

In one embodiment, the blood perfusion parameter is indicative of the heart rate of the user and/or of a blood velocity in the tissue sample. In this respect, it is known that the heart rate and the blood velocity are indicative of the physiological condition of the user, which can thus be monitored by means of the sensor device in these embodiments. In particular, irregular heart rates or blood velocities (i.e. too high or low heart rates or blood velocities) may be indicative of cardiovascular diseases of the user, such as, for example, hypertension and atherosclerosis, coronary artery disease, chronic heart failure, peripheral vascular disease, stroke, diabetes, chronic kidney failure, and infectious diseases. Moreover, an irregular heart rate or a blood velocity may be indicative of other detrimental health conditions of the user the sensor device. Thus, such diseases and detrimental health conditions can be detected by means of the sensor device. Further, the sensor device allows its user to monitor his heart rate and/or blood velocity during sporting activities and/or in other situations.

In a related embodiment, the evaluation unit is configured to estimate a frequency of changes of the contrast values and to determine the blood perfusion parameter indicative of the heart rate of the user based on the estimated frequency.

In a further embodiment, the evaluation unit is configured to substantially continuously compare the blood perfusion parameter with at least one threshold and to control the optical sensor device to execute an alarm routine, if the blood perfusion parameter exceeds or falls below the threshold. In particular, the evaluation unit may be configured to compare the blood perfusion parameter with the threshold in regular time intervals.

The threshold may particularly be set such that values of the blood perfusion parameter larger than the threshold are indicative of a detrimental health condition of the user of the sensor device. By initiating the alarm routine in case the blood perfusion parameter exceeds such a threshold, the sensor device can particularly indicate such a detrimental health condition so that suitable measures can be taken. For this purpose, the alarm routine may particularly comprise an output of an acoustic and/or visual warning indication by the sensor device. Further, a low heart rate or blood velocity may be indicative of a detrimental health condition of the user. In this respect, the evaluation may be configured to execute an alarm routine in case the blood perfusion parameter falls below a suitably selected threshold. In a further implementation, the evaluation unit may be configured to compare the blood perfusion parameter with two thresholds and may initiate an alarm routine, if the blood perfusion parameter exceeds a first threshold or if the blood perfusion parameter falls below a second threshold. Preferably, the second threshold is smaller than the first threshold.

In one embodiment in which the blood perfusion parameter is indicative of a blood velocity in the tissue sample, the sensor device further comprises a temperature sensor, and the evaluation unit is configured to prevent the initiation of the alarm routine when it determines that a temperature measured using the temperature sensor decreases by an amount exceeding a predefined threshold within a predetermined period of time. This embodiment takes account of the fact that the blood perfusion usually increases when the temperatures suddenly drops down by a greater amount, as it is for example the case when the user moves from a warm indoor environment to a cold outdoor environment. In particular, the initiation of an alarm routine may be prevented which is initiated in case the blood velocity exceeds a threshold. By preventing the initiation of such an alarm routine in the aforementioned situation, false alarms resulting from a temperature-dependent increase of the velocity parameter can be prevented.

In a further embodiment in which the blood perfusion parameter is indicative of a blood velocity in the tissue sample, the sensor device further comprises an altimeter and the evaluation unit is configured to determine the threshold on the basis of an altitude determined using the altimeter. This embodiment takes account of the fact that the level of blood perfusion is typically higher in greater altitudes. When the evaluation unit initiates an alarm in case the blood velocity exceeds a threshold, this threshold is preferably increased with an increasing altitude. Hereby, it is particularly possible to prevent false alarms resulting from an altitude-dependent increase of the blood velocity. Moreover, in case the evaluation unit initiates an alarm if the blood velocity falls below a threshold, this threshold may likewise be increased with an increasing altitude in order to improve the sensitivity of the sensor device with respect to low blood velocities in greater altitudes.

In a further embodiment in which the blood perfusion parameter is indicative of a blood velocity in the tissue sample, the sensor device further comprises a pressure sensor for measuring a pressure applied by the sensor device to the tissue sample and the evaluation unit is configured to detect changes of the pressure and to control the sensor device to output a corresponding information when a change of the pressure is detected. This embodiment takes account of the fact that the level of blood perfusion is typically lower when the sensor device applies a higher pressure on the tissue sample. Thus, measurements of the blood perfusion parameter made for different pressures are usually not comparable with each other. Therefore, the evaluation unit controls the sensor device to output a corresponding information about the pressure change to the user of the sensor device. On the basis of this information, the user may adjust the pressure in such a way that the original pressure is established again.

If the evaluation unit compares the blood perfusion parameter with the aforementioned threshold(s), the evaluation unit may also adapt the threshold(s) based on the detected pressure change in addition or as an alternative to the output of the information about the pressure change. In case the evaluation unit initiates an alarm routine in case the blood velocity exceeds a threshold, this threshold may particularly be decreased with an increasing pressure. Hereby, a sufficient sensitivity of the sensor device can be ensured in case the sensor device applies a high pressure on the tissue sample. In case the evaluation unit initiates an alarm routine if the blood velocity falls below a threshold, this threshold may likewise be decreased with an increasing pressure in order to prevent false alarms.

In one embodiment, the blood perfusion parameter is indicative of a blood velocity in the tissue sample, and the sensor device further comprises a position sensor for detecting a displacement of the sensor device relative to the tissue sample, the evaluation unit being configured to control the sensor device to output a corresponding information when a displacement of the sensor device relative to the tissue sample is detected.

Hereby, account can be taken of the fact that the values of the blood perfusion parameter usually depend on the measurement location, particularly because the tissue composition usually differs depending of the measurement location. Thus, measurements of the blood perfusion parameter after the displacement are not directly comparable with the measurements performed at the original position of the sensor device. Therefore, the evaluation unit controls the sensor device to output a corresponding information to the user of the sensor device so that the user can take into account the changed conditions under which the measurements of the blood perfusion parameter are made. In case the evaluation unit compares the blood perfusion parameter with the aforementioned threshold, this comparison may be interrupted in case a displacement of the sensor device is detected in addition or as an alternative to the output of the information about the displacement of the sensor device.

In a related embodiment, the evaluation unit may be configured to control the sensor device to output information to the user of the sensor device, which is indicative of the reverse direction of the detected displacement. On the basis of such information the user can be instructed to re-position the sensor device at the original position, i.e. the position before the displacement occurred.

The position sensor may particularly include a camera capturing images of the user's skin and the evaluation unit may be configured to recognize a characteristic pattern formed by skin irregularities, such as, for example, freckles or birthmarks. In this embodiment, the evaluation unit may detect a displacement of the sensor device relative to the tissue sample when the position of the characteristic pattern within an image captured by the camera differs from the position of the pattern in a previously captured image. Moreover, the evaluation may be configured to determine the reverse direction of the displacement of the pattern in the images in order to control the sensor device to output a corresponding information as explained above.

In one embodiment, the evaluation unit is configured to determine plural values of the blood perfusion parameter according to different tissue depths on the basis of the separate contrast values. Hereby, it is particularly possible to asses the velocity distribution of the blood within different layers of the tissue sample.

In order to eliminate motion artifacts, a related embodiment provides that the evaluation unit is configured to determine the motion-corrected value of the blood perfusion parameter based on the values of the blood perfusion parameter according to the different tissue depths. The motion-corrected value of the blood perfusion parameter may particularly be calculated as a linear combination of the values of the blood perfusion parameter corresponding to different tissue depths.

In one embodiment, the second opening of the contact surface of the housing, through which scattered light enters the housing, is configured as an aperture, the size of the aperture being selected such that at least some speckles of the speckle pattern have a predetermined minimum size. In a related embodiment, the a plurality of detection elements is associated with at least one tissue depth and the light detection unit is configured to capture an image of the speckle pattern, the image comprising a plurality of pixels corresponding to the detection elements, and the size of the aperture is selected such that a speckle having the minimum size covers at least two detection elements of the plurality of detection elements. Hereby, it can be ensured that changes of the speckle contrast can be detected by means of the light detection unit. If the speckles were smaller than one of the detection elements or if the speckle size would approximately correspond to the size of one of the detection elements, many of such changes would not be visible in the images captured by the light detection unit.

In a further aspect, the invention suggests a method for determining at least one blood perfusion parameter of a user. The method comprises:
providing a sensor device comprising a light source for providing coherent light for scattering in a tissue sample of the user and a light detection unit for receiving at least part of the scattered coherent light, the light source and the light detection unit being arranged relative to each other in a re-emission geometry and the light detection unit comprising plural light detection elements for capturing light intensity values in accordance with a speckle pattern formed by scattered coherent light, the light detection elements being arranged in increasing distances from the light source and being associated with different tissue depth;
bringing a contact surface of a housing of the sensor device into contact with the tissue sample, the contact surface comprising a first opening through which light emitted by the light source can leave the housing and a second opening through which scattered light captured the light detection unit can enter the housing;
determining separate contrast values for the light detection elements based on the captured light intensities, and
determining one motion-corrected value of the blood perfusion parameter based on the contrast values associated with the different tissue depths.

It shall be understood that the optical sensor device of claim 1 and the method of claim 14 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily components of one embodiment of an optical sensor device for determining at least one blood perfusion parameter of a user,

FIG. 2a shows exemplarily an image of a speckle pattern captured using an aperture having a diameter of 1 mm,

FIG. 2b shows exemplarily an image of a speckle pattern captured using an aperture having a diameter of 2.8 mm,

FIG. 3 shows schematically and exemplarily a detection of light propagating through a tissue sample via different propagation paths running in different tissue depths,

FIG. 4 shows schematically and exemplarily components of one embodiment of the optical sensor device comprising an altimeter,

FIG. 5 shows schematically and exemplarily components of one embodiment of the optical sensor device comprising a temperature sensor,

FIG. 6 shows schematically and exemplarily components of one embodiment of the optical sensor device comprising a pressure sensor, and

FIG. 7 shows schematically and exemplarily components of one embodiment of the optical sensor device comprising a position sensor configured as a camera.

### DETAILED DESCRIPTION OF EMBODIMENTS

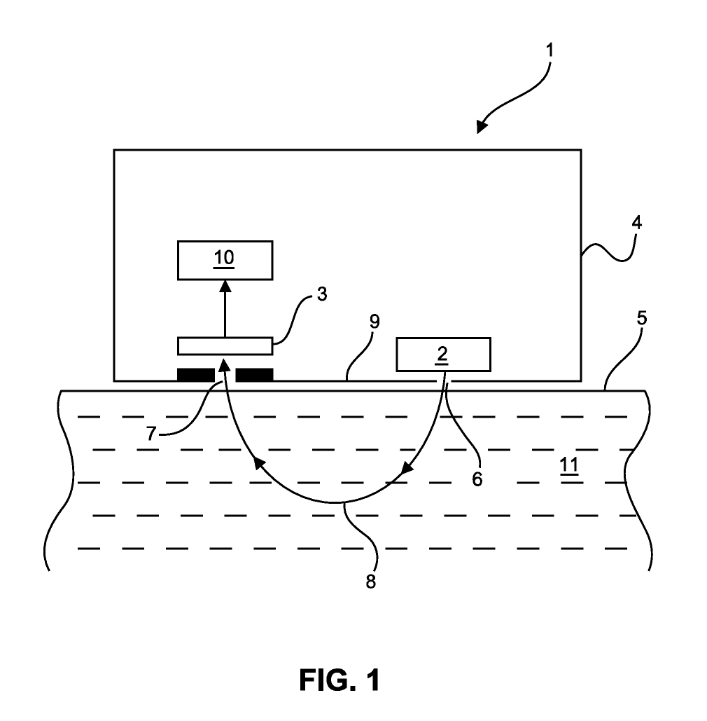

FIG. 1 shows schematically and exemplarily components of an optical sensor device 1 for determining one or more blood perfusion parameter(s) of a user of the sensor device 1. As will be further explained herein below, the blood perfusion parameter may correspond to the heart rate of the user. In addition or as an alternative, the sensor device 1 may be capable to determine a blood perfusion parameter indicative of the velocity of the blood flowing through a tissue sample 11 of the user.

The sensor device 1 may be a portable device, which is worn by its user on a suitable part of his body during operation. In particular, the user may wear the sensor device 1 at one extremity of his body, specifically on the wrist or on one finger or toe. The wearing of the sensor device 1 at an extremity of the user's body is preferred especially for determining the heart rate, because the differences in the level of blood perfusion, which can be detected in the sensor device 1, are more noticeably at the extremities of the body.

The sensor device 1 may be worn by the user substantially continuously or during longer periods of time in his everyday life in order to monitor one or more blood perfusion parameter(s). In particular, the sensor device 1 may be worn by persons having an increased risk of detrimental health conditions, such as, e.g. elderly persons. Such detrimental health conditions can early be detected using the sensor device 1 so that suitable measures can early be taken. Likewise, the sensor device 1 may be used in order to monitor blood perfusion parameters, such as e.g. the heart frequency, during sporting activities or the like.

The sensor device 1 may be configured such that it can be carried at the user's wrist. In particular, the sensor device 1 may be configured in form of a wrist watch in specific embodiments. This allows the user to carry the sensor device 1 in a convenient way. In these embodiments, the sensor device 1 may be a stand-alone device substantially having the form of a wrist watch. Or, the sensor device 1 may be integrated into a so-called smart watch, which includes the sensor device 1 and which further includes additional components for one or more further functions.

The sensor device 1 determines the blood perfusion parameter(s) on the basis of laser speckle imaging. In order to perform speckle imaging, the sensor device 1 comprises a light source 2 for emitting coherent light and a light detection unit 3 for collecting part of the light after having been scattered within the tissue sample 11. Both the light source 2 and the light detection unit 3 are included in a housing 4 of the sensor device 1. Within the housing 4, the light source 2 and the light detection unit 3 are arranged relative to each other in a re-emission geometry. This does particularly mean that the light source 2 and the light detection unit 3 are arranged relative to each other such that the tissue sample 11 is not positioned between the light source and the light detection unit. Such a re-emission geometry does particularly allow for constructing a compact sensor device 1 which can easily be carried by its user.

More specifically, the housing 4 may be placed onto the skin 5 of the user in such a way, that the housing 4 is in contact with the skin 5 in a region 9 of the housing 4 which is referred to as contact surface herein. In the area of the contact surface 9, the housing 4 disposes of an opening 6 through which coherent light emitted by the light source 2 leaves the housing 4 and penetrates the tissue sample 11 beneath the user's skin 5. The light source 2 is arranged near in the opening 6 in such a way that emitted light rays traverse the opening with a certain angle relative to the contact surface. The light detection unit 3 is preferably arranged adjacent to the light source 2 within the housing 4. In particular, the light detection unit 3 is arranged in the area of a further opening 7 in the contact surface 9, which is arranged adjacent to the opening 6 and through which part of the scattered coherent light enters into the housing 4 and hits the light detection unit 3.

When the user carries the sensor device 1 with the contact surface 9 of the housing 4 contacting the user's skin 5, relative movements of the tissue sample 11 and the sensor device 1 can be minimized. Hereby, the reliability of the measurements of the sensor device 1 can be improved since relative movements of the tissue sample 11 and the sensor device 1 would lead to undesired motion artifacts in the measurements. Moreover, the reflection of light at the user's skin 5 can be minimized. In order to affix the sensor device 1 in such a way, suitable fastening means may be provided. When the sensor device 1 has the form of a wrist watch, the fastening means may be an adjustable belt which is affixed to the housing 4 of the sensor device 1 and which closely surrounds the user's wrist together with the housing 4 in order to hold the housing 4 in place.

In order to further minimize the reflection of the light at the skin 5, an optical coupling material may be used in such a way that it fills the cavity between the light source 2 and the skin 5. This optical coupling material may be a suitable gel, which may be applied by the user to the user's skin 5 in the area of the contact surface 9, before attaching the sensor device 1.

The light source 2 is a laser device emitting coherent light particularly in the red spectral range. Thus, there is a high scattering probability for the light being scattered at red blood cells within the tissue sample 11. Preferably, the light source 2 is configured as a semiconductor laser diode emitting in the suitable spectral range. In particular, the light source 2 may be a so called vertical-cavity surface-emitting laser (VCSEL) in which the laser light is emitted perpendicular to the wafer surface. However, the light source 2 may also be configured in another way. For instance, it may be configured as an edge-emitting laser device.

During operation of the sensor device 1, light emitted by the light source 2 penetrates the skin 5 after having traversed the opening 6 and is scattered by the red blood cells within the tissue sample 11. The light detection unit 3 collects scattered light which is re-emitted by the tissue sample 11 in the area of the opening 7 of the contact surface 9. This is schematically and exemplarily shown in FIG. 1 which illustrates the propagation of one scattered light ray 8 through the tissue sample 11. Due to interference of the light scattered from the randomly distributed red blood cells, the scattered light produces a random pattern which is usually called speckle pattern. In the sensor device 1, the light detection unit 3 measures light intensity values in accordance with the speckle pattern produced by the interfering scattered light having traversed the opening 7 in the housing 4 of the sensor device 1 and reaching the light detection unit 3. For this purpose, the light detection unit 3 disposes of at least one light-sensitive detection element for determining the intensity of the light collected by the light detection unit 3.

The light intensity values determined by the detection unit 3 are evaluated in an evaluation unit 10 of the sensor device 1. The evaluation unit 10 is preferably integrated into the housing 4 of the sensor device 1 in addition to the light source 2 and the light detection unit 3, and may be configured as a microprocessor including a processing unit and a memory for storing data. For evaluating the measurements provided by the light detection unit 3, the evaluation unit 10 comprises a corresponding software program which is stored in the memory of the evaluation unit 10 and executed in the processor during the operation of the sensor device 1.

The evaluation of the measured speckle patterns in the evaluation unit 10 is preferably made on the basis of changes of the speckle patterns determined in the evaluation unit 10. Such changes are due to the movement of the red blood cells within the tissue sample 11, by which the light emitted by the light source 2 is scattered. Therefore, these changes allow for determining parameters relating to the blood perfusion within the tissue sample. In particular, the evaluation unit 10 may perform the evaluation of the images on the basis of the contrast of the speckle patterns in accordance with a so-called laser speckle contrast analysis (LASCA).

For this purpose, the evaluation unit 10 preferably calculates contrast values on the basis of the measurements performed using the light detection unit 3. Each calculated contrast value is indicative of the amount of variation in the measured intensity distribution relative to an average intensity. In particular, the speckle contrast K may be calculated as K=σ/<I>, where σ is a standard deviation of intensity values and <I> is an average of these intensity values. In one variant, one contrast value may be calculated on the basis of intensity values measured at different locations across a detection surface of the light detection unit substantially at the same point in time. Such a contrast value is also referred to as spatial contrast value hereinafter. In a further variant, one contrast value, which is also referred to as temporal contrast value hereinafter, may be calculated on the basis of intensity values measured at consecutive points in time at the same location. In particular, one temporal contrast value may be calculated on the basis of intensity values measured during a time period of a predefined length. In the next time period, a new temporal contrast value may be calculated.

In one embodiment, the light detection unit 3 includes an array of detection elements covering a detection surface of the light detection unit and capturing images including a number of pixels corresponding to the detection elements. Such a light detection unit 3 will also be referred to as image sensor herein. In one exemplary implementation, the image sensor may be configured as a charge-coupled device (CCD) image sensor. However, other configurations of the image sensor are likewise possible. The detection elements of the image sensor simultaneously capture light intensity values corresponding to the speckle pattern formed on the detector surface. Preferably, these light intensity values are captured quasi-continuously in accordance with a certain image or frame rate. On the basis of some or all of these simultaneously measured light intensity values, the evaluation unit 10 may calculate one spatial contrast value for each image or frame. As will further be explained below, it is likewise possible to define groups of pixels and to calculate one spatial contrast value for each of these groups. Moreover, it is in principle also possible to calculate temporal contrast values for at least some of the detector elements individually.

In a further embodiment, the light detection unit 3 may comprise a single detection element, such as, for example, a photodiode, which captures light intensity values at preferably regular time intervals. Using such a light detection unit 3, temporal contrast values can be determined in the way described above.

Preferably, the light detection unit 3 in the aforementioned embodiments detects unfocused scattered light re-emitted by the tissue sample 11. Thus, the sensor device 1 does not include lenses or other optical elements for focusing the collected scattered light on the light detection unit 3. This allows for providing a compact sensor device 1 that can easily be carried by the user.

However, it has been found that it may not be possible to properly detect and evaluate the unfocused speckle patterns, when no measures for influencing the optical characteristic of the detected speckle patterns are taken at all. In particular, the size of the patterns may be too small so that it is not possible to properly detect individual speckles by means of the detection elements, when the light detection unit 3 is configured as an image sensor and spatial contrast values are determined on the basis of the measurements performed by the image sensor. In order to improve the detection of the speckle pattern by means of the light detection unit, the opening 7 in the housing 4, through which the scattered light travels to the light detection unit 3, may be configured as an aperture having a defined size. In one implementation, a circular aperture having a defined diameter is used. However, it is likewise possible to use an aperture having a different shape.

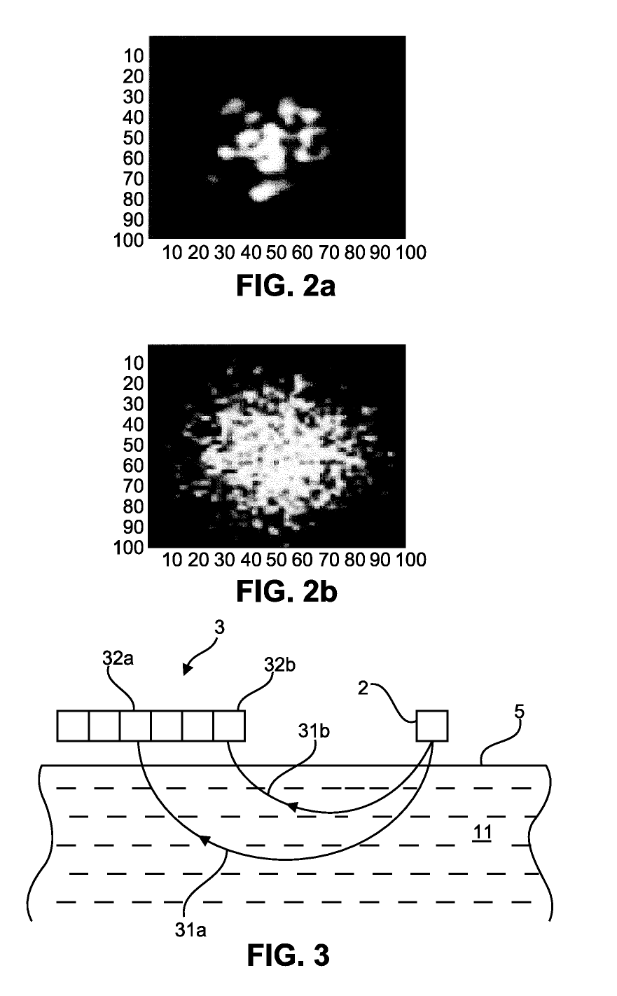

In this respect, it has been found that the size of the aperture determines the size of the speckles (i.e. the size of the spots with a high light intensity) in the images captured by the light detection unit 3. In particular, it has been found that the speckle size increases with a decreasing size of the aperture. This finding is also illustrated in FIGS. 2a and 2b: These figures show images of the speckle pattern captured using sensor devices 1 which were configured in the above-described manner and which comprised apertures 7 having a diameter of 1 mm (FIG. 2a) and 2.8 mm (FIG. 2b). As will be appreciated from the figures, the speckles in the pattern captured using the sensor device 1 having the smaller aperture are larger compared with the speckles in the pattern captured using the sensor device 1 having the larger aperture.

On the basis of these findings, the size of the aperture 7 is preferably selected such that at least most speckles in the captured speckle patterns cover a plurality of pixels or detection elements of the light detection unit 3. Hereby, the speckle contrast determined on the basis of the images can be increased and, at the same time, the contrast differences resulting from differences in the blood velocity are increased. The selection of a suitable aperture size resulting in sufficiently large speckles can be made on the basis of test experiments or simulations for different aperture sizes.

In one embodiment, the evaluation unit 10 determines the blood perfusion parameter(s) on the basis of one spatial or temporal contrast value, which may be estimated based on light intensity values measured at the same point in time using an image sensor or measured at consecutive points in time using a single light detection element as discussed above. However, parts of the tissue sample 11 covered by the sensor device 1 may move relative to the sensor device 1 due to motions of the user. Particularly when the sensor device 1 is worn at the user's wrist, parts of the tissue sample 11 may move relative to the sensor device due to movements of the user's hand and/or fingers, for example. Such movements within the tissue sample 11 are due to the fact that the tissue in the area of the wrist is interconnected with the tissue in the area of the hand and fingers. Moreover, tendons or the like, which move in case of a movement of the hand and/or fingers, may run through the tissue sample 11.

Movements of this kind typically lead to motion artifacts in the determination of the blood perfusion parameter, since a reduction of the speckle contrast due to such movements may erroneously by treated as an indication of an increased blood perfusion. In order to eliminate or reduce such motion artifacts, the evaluation unit 10 may determine plural contrast values for different depths in the tissue sample 11 and may determine the blood perfusion parameter on the basis of these plural contrast values. This approach is based on the observations that movements of the aforementioned kind do typically not lead to a homogenous motion of the complete tissue sample 11. Rather, such movements typically affect different layers of the tissue sample 11 to a different extent. Therefore, it is possible to perform a correction of the motion artifacts on the basis of plural contrast values determined for different tissue depths.

The determination of the contrast values for different tissue depths exploits the fact that the light travels through the tissue from the light source to the light detection unit 3 substantially in a banana-shaped path as schematically shown for two paths 31a and 31b in FIG. 3. This form of the light propagation paths ensues from multiple scattering of the photons within the tissue. As a result of this form of the light propagation path, scattered light detected in a greater distance from the light source 2 has traversed deeper layers of the tissue sample 11 than scattered light detected in a smaller distance from the light source 2. Thus, speckle patterns of light detected closer to the light source 2 and speckle patterns of light detected farther away from the light source 2 are influenced by motions in different depths of the tissue.

This is also illustrated in FIG. 3 in which it can be appreciated that the light propagating along the path 31a has traversed deeper layers than the light propagating along the path 31b. Here, the light propagating along the path 31a is collected by a detection element 32a and the light propagating along the path 31b is collected by a detection element 32b of the light detection unit 3. The detection element 32a has a first distance from the light source 2 and the detection element 32b has a second distance from the light source 2, which is smaller than the first distance. In addition, the light detection unit 3 may comprise further detection elements for detection light in one or more further distances from the light source 2 as shown in FIG. 3. Moreover, as explained above, there may be a group of detection elements which is associated to one distance from the light source (and, thus, to a certain tissue depth) for each distance instead of a single detection element as shown in FIG. 3.

The evaluation unit 10 may determine plural contrast values for speckle patterns detected in different distances from the light source 2. Such contrast values are also referred to as depth-related contrast values hereinafter in order to distinguish them from contrast values which are not related to certain depths and which are also referred to as general contrast values hereinafter. In order to determine depth-related contrast values, light intensity values which are measured at different distances from the light source 2 are evaluated separately. In particular, one contrast value is determined for each of a plurality of predefined distance ranges on the basis of light intensities measured in each distance range. For this purpose, one or more detection element(s) of the light detection unit 3 are assigned to each distance range as explained above. In one embodiment, a single detection element having a distance corresponding to the respective distance range is assigned to each distance range. In this case, the evaluation unit 10 may calculate separate temporal contrast values for each distance range. In a further embodiment, a group of detection elements having distances in the corresponding distance range is assigned to each distance range. In this implementation, the evaluation unit 10 may determine a spatial contrast value for each distance range on the basis of the light intensities measured by means of the associated detection elements. As explained above, these contrast values are indicative of the blood perfusion in different depths of the tissue sample 11.

On the basis of the depth-related contrast values, the evaluation unit 10 may determine corresponding values of the blood perfusion parameter. Moreover, as explained above, the evaluation unit 10 may perform a correction of motion artifacts on the basis of plural contrast values determined for different tissue depths. In a related embodiment, the evaluation unit 10 determines one single contrast value which is also referred to as motion-corrected contrast value on the basis of the depth-related contrast values. In an alternative embodiment, the evaluation unit 10 determines one value of the blood perfusion parameter for each of the contrast values associated with the different tissue depths and estimates a single motion-corrected value for the blood perfusion parameter on the basis of the individual values of the blood perfusion parameter.

The correction of the motion artifacts may be made on the basis of the assumption that the motion artifacts are smaller for blood vessels located in a smaller tissue depth. This assumption is based on the finding that the amplitude of the motion artifacts is approximately inversely proportional to the blood pressure and that the blood pressure is lower in the smaller blood vessels close to the surface of the skin 5. Thus, the motion artifacts will be more pronounced for these blood vessels. On this basis, the correction of the motion artifacts may be made by means of a linear superposition of measurement values (i.e. contrast values or values of the blood perfusion parameter) for different depths.

In particular, the correction is made on the basis of measurement values for different depths acquired at successive points in time. From the successively measured values for each depth, the evaluation unit may generate one vector, respectively. Further, the evaluation unit 10 may combine the vectors by subtracting the vector including the measurement values for one depth from the vector including the measurements values for a further depth multiplied by a factor. Then, the evaluation unit 10 determines the factor value for which the combined vector has a minimal standard deviation. This determination may be made on the basis of an iterative procedure, for example. The resulting vector, i.e. the combination of vectors generated using the determined factor value, may correspond to the motion-corrected vector including the motion corrected measurement values for the successive points in time.

As mentioned above, the evaluation unit 10 may particularly determine the heart rate of the user on the basis of changes of the contrast value determined in accordance with the measurements of the light detection unit 3. In this respect, it is to be noted that each heart contraction accelerates the blood in the blood vessels so that the red blood cells achieve a relatively high velocity. This higher velocity leads to a lower speckle contrast due to the blurring of the speckle images caused by the movement of the red blood cells. After a heart contraction, the velocity decreases during a period of the heart motion until it is accelerated again by the next heart contraction and, as a consequence of the decreasing velocity, the speckle contrast increases. Thus, the speckle contrast decreases with a relative high gradient and increases again with a lower gradient between two heart contractions. Hence, the speckle contrast varies periodically (following the periodic variations of the blood velocity) and the frequency of the variation of the speckle contrast corresponds to the heart frequency.

Following these observations, the evaluation unit 10 may determine the frequency of the periodic variation of the speckle contrast calculated for successive images and may output this frequency as an estimate for the heart frequency of the user. For determining the frequency of the variation of the speckle contrast, any known procedure for determining the frequency of a periodically varying parameter may be used. For instance, the evaluation unit may determine the periods of the variation of the speckle contrast, and may estimate the heart frequency on the basis of the determined periods. As explained above, this evaluation is preferably made on the basis of motion-corrected contrast values, or the evaluation unit may determine individual heart rate values for different tissue depths on the basis of the corresponding depth-related contrast values and may then estimate the user's heart rate based on these heart rate values. Hereby, it can be prevented that the determination of the heart rate is affected by contrast changes resulting from user motion instead of blood perfusion due to the motion of the heart. However, it is likewise also possible to determine the heart rate on the basis of general contrast values which are not corrected with respect to the above-described motion artifacts.

The estimated heart frequency may be visually output to the user on a display unit of the sensor device 1 (not shown in the figures). Further, the evaluation unit 10 preferably compares the estimated heart frequency with a predetermined upper threshold and/or a predetermined lower threshold. The upper and lower threshold values may be selected such that heart frequencies above the upper threshold and below the lower threshold are likely indicative of an impairment of the user's health. In addition or as an alternative, the upper and/or lower thresholds may be defined by the user of the sensor device 1 in another way. In particular, the user may configure the upper and/or thresholds in a certain way in order to perform a performance control during sporting activities.

When at least one upper and/or lower threshold is configured in the sensor device 1, the evaluating unit 10 compares the estimated heart frequency values with the upper and/or lower thresholds. In case the evaluation unit 10 determines that the estimated heart frequency is below a configured lower threshold and/or in case the evaluation unit 10 determines that the estimated heart frequency is larger than a configured upper threshold, the evaluation unit 10 may initiate an alarm routine. In one embodiment, the alarm routine may comprise an output of a visual and/or acoustic alarm signal by the sensor device 1.

In a further embodiment, the sensor device 1 determines a blood perfusion parameter indicative of the blood velocity in the tissue sample 11 in addition or as an alternative to the user's heart frequency. The blood perfusion parameter indicative of the blood velocity—which is also referred to as velocity parameter herein—is preferably determined in such a way that it increases when the speckle contrast decreases. In particular, the velocity parameter may be calculated based on the speckle contrast as 1/K².

Similar to the heart rate, the velocity parameter is preferably determined on the basis of motion-corrected contrast values, or the evaluation unit 10 may determine individual velocity parameter values for different tissue depths on the basis of the corresponding depth-related contrast values and may then estimate the velocity parameter based on these individual values for the velocity parameter. Hereby, motion artifacts affecting the determined velocity parameter can be reduced. However, it is likewise also possible to determine the velocity on the basis of general contrast values which are not corrected with respect to motion artifacts.

In a further embodiment, the evaluation unit 10 may determine individual values of the velocity parameter for different tissue depths on the basis of the contrast values measured for such tissue depths. These values of the velocity parameter may be output by the sensor device 1 in order to provide a three-dimensional velocity map for the tissue sample.

Preferably, the evaluation unit 10 determines mean values of the velocity parameter in substantially regular time intervals. For this purpose, the evaluation unit 10 may calculate mean values for successive time periods, where each mean value may be calculated from the values of the velocity parameter determined during the respective time period. The time periods are preferably selected such that they at least include two or more heart beats, since the blood velocity varies periodically as a function of the heart motion as explained above.

Preferably, the evaluation unit 10 controls the sensor device 1 to output an information about the determined mean values of the velocity parameter at a display of the sensor device 1. This information may include the absolute values of the determined mean values. However, the absolute value of the velocity parameter may not be meaningful to the user of the sensor device 1. Therefore, relative values with respect to a reference value may be output by the sensor device 1. These relative values may include percentages of the determined mean values with respect to the reference value or differences between the determined mean values and the reference value. The reference value may be determined by the evaluation unit 10 based on one or more measurements of the velocity parameter. These measurements may be performed when the user is in good health condition and when the sensor device 1 is operated in normal environmental conditions, i.e. in such environmental conditions in which it is usually operated by its user. For performing the measurements, the sensor device 1 may dispose of a special mode of operation which may be activated by the user when the aforementioned conditions apply. When one mean value of the blood velocity parameter is determined in this mode of operation, the evaluation unit 10 may store this mean value as the reference value. In case plural mean values of the blood velocity parameter are determined in the aforementioned mode of operation, the evaluation unit may e.g. determine a mean of these values and may store this mean as the reference value.

Further, the determined mean values of the blood velocity parameter may be compared with an upper and/or lower threshold value (during the normal mode of operation). In one embodiment, the threshold value(s) may correspond to value(s) pre-stored in the sensor device 1. As an alternative, the threshold(s) may be determined as a predetermined multiple and/or fraction of the reference value of the velocity parameter. On the basis of the comparison between the determined mean values of the blood velocity parameter and the upper and/or lower threshold, the evaluation unit 10 may initiate an alarm routine. In particular, the alarm routing may be initiated, if the evaluation unit 10 determines that a mean value of the velocity parameter is larger than the upper threshold value. In addition or as an alternative, the evaluation unit 10 may initiate an alarm routine in case it determines that the mean value of the velocity parameter is smaller than the lower threshold value. The alarm routine can be configured in a similar way as described above in connection with the monitoring of the user's heart frequency. Thus, the alarm routine may comprise that the sensor device 1 outputs an acoustic and/or visual warning indication under the control of the evaluation unit 10.

The comparison between the mean value of the velocity parameter and the upper threshold may particularly be made when the sensor device 1 is used for monitoring the user's health condition during sporting activities. In this case, an excessive blood flow indicated by a mean value above the upper threshold is indicative of a detrimental health condition of the user of the sensor device 1. The comparison between the mean value of the velocity parameter and the lower threshold may particularly be made when the sensor device 1 is used for monitoring the health status of users having a defective epithelial function. For such users, a mean value of the blood velocity parameter which is smaller than a properly selected lower threshold may be indicative of an insufficient epithelial function, and therefore an alarm routine may be initiated in case the mean value falls below the lower threshold.

In embodiments of the sensor device 1, in which the evaluation unit 10 estimates the velocity parameter, the sensor device 1 may additionally comprise one or more further sensor(s), which monitor environmental conditions influencing the velocity parameter. When the evaluation unit 10 detects a change of the environmental conditions which influences the velocity parameter, it may control the sensor device 1 to output a corresponding information to the user of the sensor device 1. Thus, the user can take the change of the environmental condition into account when evaluating the measurements of the blood velocity parameter.

In case the evaluation unit compares the blood velocity parameter with an upper and/or lower threshold, it may additionally or as an alternative adapt the threshold(s) to different environmental conditions on the basis of the measurements performed by the additional sensor(s). In particular, the evaluation unit 10 may increase the upper threshold when the sensor(s) indicates that the sensor device 1 is operated in a condition which is connected with an increased velocity parameter compared to a condition on the basis which the threshold value is selected. By such an adaptation of the threshold, it is particularly possible to prevent false alarms when an increased velocity parameter results from a certain environmental condition rather than a detrimental health condition of the user of the sensor device 1. Further, the evaluation unit 10 may increase the lower threshold value when the sensor(s) indicate that the sensor device 1 is operated in a condition which involves an increased velocity parameter. Hereby, the sensitivity of the sensor device 1 to detrimental health conditions involving a low blood velocity can be improved.

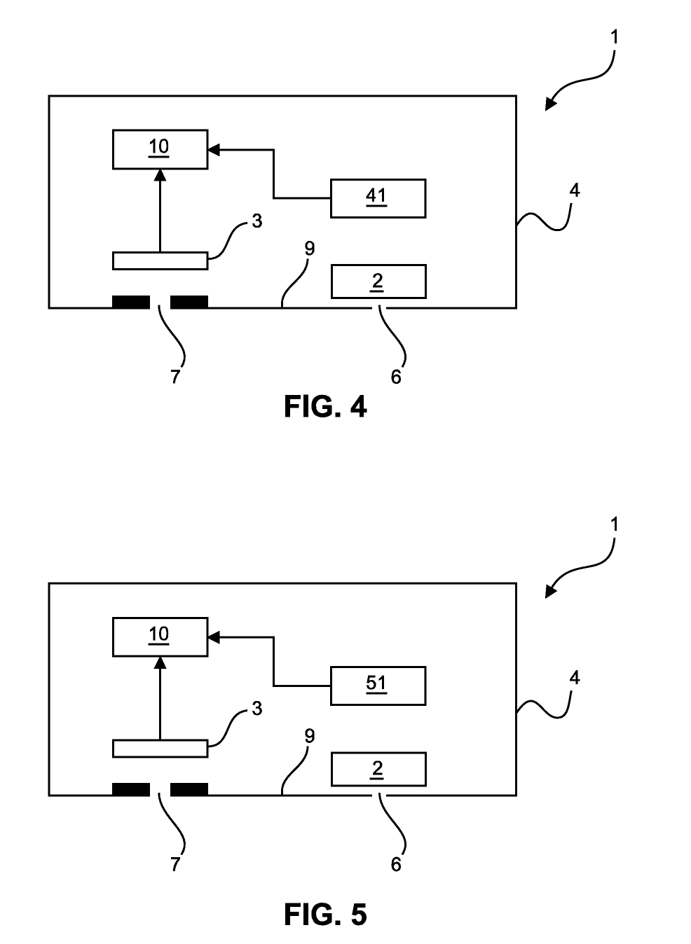

In one related embodiment, the sensor device 1 additionally includes an altimeter 41 for measuring the altitude in which the sensor device 1 is being operated. This embodiment is schematically and exemplarily illustrated in FIG. 4. The altimeter 41 may be configured in any suitable way known to the person skilled in the art. The measured altitude values may be taken into account due to the fact that the level of blood perfusion is typically higher in greater altitudes.

On the basis of the measured altitude, the evaluation unit 10 may determine the upper and/or lower threshold value(s) to be compared with the velocity parameter. In particular, the evaluation unit 10 may increase the upper threshold value when the measured altitude increases and decrease the upper threshold value when the measured altitude decreases. For this purpose, the evaluation unit 10 may increase (in case of an increased altitude) or decrease (in case of a decreased altitude) the threshold relative to a base threshold value by amounts determined based on a difference between the measured altitude and an altitude assigned to the base threshold value. The base threshold value and an associated altitude may be pre-stored in the sensor device 1. Or, the base threshold value is determined on the basis of a reference value of the velocity parameter selected by the user. To this base threshold value, the evaluation unit 10 may assign the altitude which is measured by the altimeter 41, when the reference value of the velocity parameter is determined. Similarly, the evaluation unit 10 may increase the lower threshold value when the measured altitude increases and decrease the lower threshold value when the measured altitude decreases.

In addition or as an alternative to the adaptation of the threshold(s), the evaluation unit 10 may control the sensor device 1 to output a corresponding information to the user of the sensor device 1, when the absolute value of the difference between the measured altitude and a reference altitude is larger than a threshold. Preferably, the reference altitude correspondence to the altitude measured during the measurement of the reference value for the blood velocity parameter. On the basis of the information about the change of the altitude with respect to the reference altitude, the user of the sensor device 1 may take the altitude change into account when evaluating the measured values of the blood velocity parameter.

Further, as schematically and exemplarily illustrated in FIG. 5, the sensor device 1 may comprise a temperature sensor 51 in addition or as an alternative to the altimeter 41. If the temperature sensor 51 is present, the evaluation unit 1 preferably monitors the measured temperature signal in order to determine situations in which the temperature decreases by an amount exceeding a predefined threshold within a predetermined period of time. The period of time is selected relatively small in order to able to detect situation in which a sudden temperature drop occurs. Thus, it is possible to detect situations in which the temperature suddenly drops by a greater amount, as it is for example the case when the user moves from a warm indoor environment to a cold outdoor environment during winter. In such situations, the blood perfusion usually significantly increases.

When the evaluation unit 10 detects such a situation, it does preferably control the sensor device 1 to output a corresponding acoustic and/or visual information in order to inform the user of the sensor device 1, that the measured values of the blood perfusion parameter are currently influenced by the temperature change. In case the evaluation unit 10 compares the blood velocity parameter with the aforementioned upper threshold, it may suspend this comparison for a predetermined time interval, when a sudden temperature change is detected in the way explained above, or it may block the initiations of the alarm routine for the predetermined time interval in this situation.

By suspending the comparison between the velocity parameter and the upper threshold value or blocking the alarm routine, false alarms resulting from such a temperature-dependent increase of the velocity parameter can be prevented.

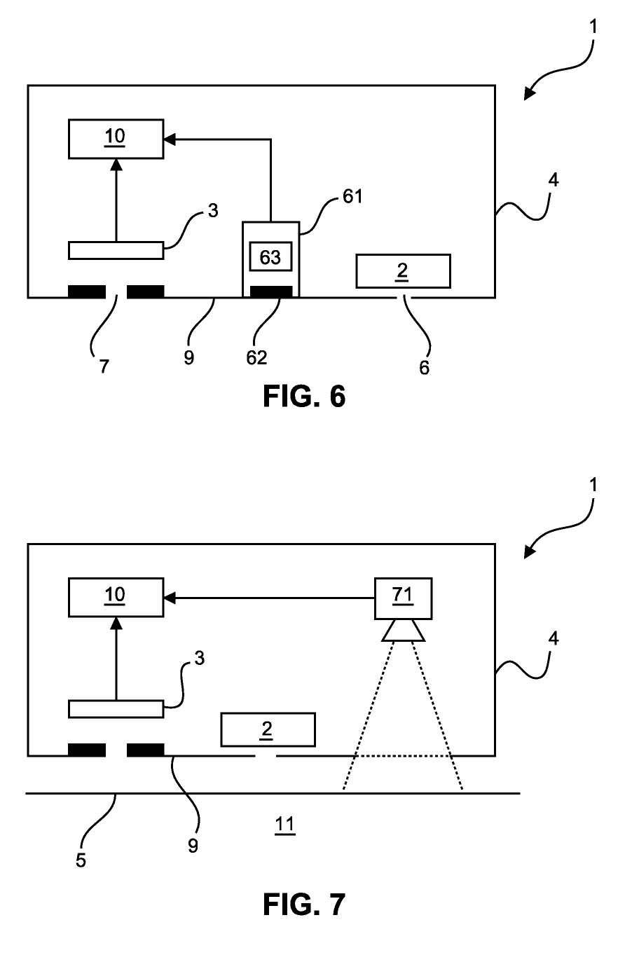

In addition or as an alternative, the sensor device 1 may comprise a pressure sensor 61 for measuring a pressure applied by the sensor device 1 to the skin 5 and the tissue sample 11 beneath the skin 5. This embodiment is schematically and exemplarily illustrated in FIG. 6. In one implementation, the pressure sensor 61 may be configured as a piezoelectric sensor. Such a sensor may comprise a piezoelectric foil 62 attached to the contact surface 9 of the housing 4 of the sensor device 1 such that pressure is applied to the piezoelectric foil when the user wears the sensor device 1. Thus, the foil provides an electric voltage from which the pressure applied to the skin 5 may be estimated by an evaluation logic 63 of the pressure sensor 61.

Typically, the level of blood perfusion is lower, when the sensor device 1 applies a higher pressure on the skin 5 of the user and the tissue sample 11 beneath the skin 5. Therefore, the evaluation unit 10 preferably monitors the measured pressure applied on the tissue sample 5 in order to detect changes of the pressure. In particular, the evaluation unit 10 may be configured to detect such a change when a difference between a measured pressure value and a pressure value measured earlier exceeds a threshold. In one implementation, the pressure value measured earlier may correspond to a reference pressure value which has been measured while the aforementioned reference value for the blood velocity parameter has been determined. In a further implementation, the evaluation unit 10 may compare each measured pressure value with the preceding pressure value in order to detect a change of the pressure applied to the tissue sample 11.

In case the evaluation unit 10 detects such a pressure change, it may control the sensor device 1 to output a corresponding information. In response to this information, the user may adjust the pressure in such a way that the pressure change is reversed. In order to assist the user in performing this adjustment, the sensor device 1 may also output an indication whether the pressure has to be increased or decreased in order to reverse the pressure change.

In such a way, the evaluation unit 10 may monitor the pressure applied to the tissue sample 11 during operation periods of the sensor device 1 in which the user continuously wears the sensor device 1. Moreover, the evaluation unit 10 does preferably detect situations in which the user newly attaches the sensor device 1 with a pressure that differs from the pressure applied on the skin 5 in a preceding wearing period of the sensor device 1.

In addition or as an alternative, the evaluation unit 10 may determine the upper and/or lower threshold value(s) to be compared with the velocity parameter on the basis of the measured pressure. In particular, the evaluation unit 10 preferably decreases the threshold value(s) with an increasing pressure and vice versa. For this purpose, the evaluation unit 10 may particularly decrease (in case of an increased altitude) or increases (in case of a decreased altitude) each threshold relative to respective base threshold value by amounts determined based on a difference between the measured pressure and a pressure value assigned to the base threshold value(s). The base threshold value(s) for the upper and/or lower threshold and an associated pressure value may again be pre-stored in the sensor device 1. Or, the base threshold value(s) for the upper and/or lower threshold may be determined on the basis of respective reference values of the velocity parameter selected by the user as described above, and the evaluation unit 10 may assign to these base threshold values the pressure which is measured by the pressure sensor, when the reference value of the velocity parameter is determined and stored.

Moreover, the measurements of the blood velocity parameter usually depend on the measurement location, particularly because the composition of the tissue usually varies with the measurement location. Therefore, the sensor device 1 may optionally include a position sensor 71, which performs measurements indicative of the position of the sensor device 1 in successive time intervals such that the measurements particularly allow for detecting displacements of the sensor device 1 with respect to the tissue sample 11. In one embodiment, a displacement of the sensor device 1 with respect to its position during the determination of the reference value for the blood velocity parameter may be determined by means of the position sensor 71. In alternative embodiments, a displacement of the sensor device 1 with respect to a preceding position at the time of a preceding measurement by the position sensor 71 may be detected.

In such a way, displacements of the sensor occurring during a wearing period may be detected. Moreover, the evaluation unit 10 may detect displacements relative to the relevant original position when the user newly attaches the sensor device 1 to the tissue sample 11 after the wearing of the sensor device 1 has been interrupted.

In case a displacement of the sensor device 1 with respect to the tissue sample 11 is detected, the evaluation unit 10 may control the sensor device 1 to output a corresponding information to the user of the sensor device 1. In response to the output of this information, the user may re-position the sensor device 1 in order to reverse the displacement. In order to prompt the user to re-position the sensor device 1, the information output by the sensor device 1 in response to the detection of displacements may include a corresponding indication. Moreover, the sensor device 1 may assist the user in re-positioning the sensor device 1. For this purpose, the evaluation unit 10 may control the sensor device 1 to output information invective of the reverse direction of the displacement. This information may e.g. include arrows pointing in the corresponding direction. On the basis of this information, the user may be guided in moving the sensor device 1 to the original position. Preferably, the information is determined for each position measurement so that the user can re-position the sensor device 1 in successive steps.

As a further option, the sensor device 1 may not be re-positioned when a displacement of the sensor device 1 with respect to the tissue sample 11 has been detected. Rather, a new reference value for the blood velocity parameter may be determined at the new position of the center device in the way already described above.

In the embodiment schematically and exemplary illustrated in FIG. 7, the position sensor 1 is configured as a camera, which is preferably included in the sensor device 1. The camera captures images of the user's skin in successive time intervals and these images are evaluated in the evaluation unit 10 in order to detect characteristic patterns formed by skin irregularities, such as, for example, freckles or birthmarks. A displacement of the sensor device 1 may be detected by the evaluation unit 10, when the position of the detected pattern within an image captured by the camera 71 differs from the position of the same pattern in a previous image, particularly in the image captured during the determination of the reference value for the blood perfusion parameter.

In order to assist the user of the sensor device 1 in a process of re-positioning the sensor device 1 at the original position as described above, the evaluation unit may determine the direction of the displacement of the characteristic pattern and may control the sensor device 1 to visually indicate the reverse direction in a suitable way. In a further embodiment, the evaluation unit may control the sensor device 1 to display an image captured at the original position of the sensor device and to display a current camera image such that it overlays the image captured at the original position such that both images can be viewed by the user at the same time. In this embodiment, the user may move the sensor device 1 in such way that the characteristic pattern in the current image superposes the characteristic pattern in the image captured at the original position.

As shown in FIG. 7, the camera 71 may be located within the housing 4 of the sensor device 1 and may be aligned such that it captures images of the user's skin within a field of view beneath the sensor device 1. In order to allow for capturing such images through the contact surface 9 of the housing 4, the contact surface 9 may be made of a translucent material, such as glass, at least in a certain area corresponding to the field of view of the camera 71. Moreover, an additional light source may be provided to illuminate the field of view of the camera (which is covered by the sensor device 1). In this embodiment, the camera 71 is preferably located such that the distance between the camera 71 and the contact surface 11 of the housing 4 of the sensor device 1 is as large as possible in order to allow the camera 71 to capture an area of the user's skin which is as large as possible.

In alternative embodiments (not shown in the figures) the camera 71 does not capture images of an area of the user's skin 5 beneath the sensor device 1 but of an area of the user's skin 5 adjacent to the sensor device 1 (when it is worn by its user). For this purpose, the camera 71 may be located in the area of an edge of the housing 4 of the sensor device 1 and may be aligned in a suitable way in this implementation, it is also possible to provide a mirror optic for guiding light originating from a certain area of the user's skin 5 to the camera 71. This facilitates the positioning of the camera 71 within the housing 4 of sensor device 1. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. An optical sensor device for determining at least one blood perfusion parameter of a user, the sensor device comprising:
a light source for providing coherent light for scattering in a tissue sample of the user; and
a light detection unit for receiving at least part of the scattered coherent light, wherein the light source and the light detection unit are arranged relative to each other in a re-emission geometry,
wherein the light detection unit comprises plural light detection elements for capturing light intensity values in accordance with a speckle pattern formed by scattered coherent light, the light detection elements being arranged in increasing distances from the light source wherein each distance is associated with a tissue depth corresponding to a tissue layer traversed by the light and wherein at least one light detection element is assigned to each distance,
wherein the sensor device comprises a housing including the light source and the light detection unit, the housing comprising a contact surface which can be brought into contact with the tissue sample and which comprises a first opening through which light emitted by the light source can leave the housing and a second opening through which scattered light captured by the light detection unit can enter the housing,
and wherein the optical sensor devices further comprises an evaluation unit configured to determines a contrast value for each distance based on the intensity captured by the at least one light detection element assigned to respective distance and to determine one motion-corrected value of the blood perfusion parameter based on the contrast values determined for the distances.

2. The optical sensor device as defined in claim 1, wherein the first and second openings are arranged relative to each other in such a way that a connection line between the first and second openings does not cross the tissue sample, when the contact surface is in contact with the tissue sample.

3. The optical sensor device as defined in claim 1, wherein the blood perfusion parameter is indicative of the heart rate of the user and/or of a blood velocity in the tissue sample.

4. The optical sensor device as defined in claim 3, wherein evaluation unit is configured to estimate a frequency of changes of the contrast values and to determine the blood perfusion parameter indicative of the heart rate of the user based on the estimated frequency.

5. The optical sensor device as defined in claim 1, wherein the evaluation unit is configured to substantially continuously compare the blood perfusion parameter with at least one threshold and to control the optical sensor device to execute an alarm routine, if the blood perfusion parameter exceeds or falls below the threshold.

6. The optical sensor device as defined in claim 5, wherein the blood perfusion parameter is indicative of a blood velocity in the tissue sample, wherein the sensor device further comprises a temperature sensor, and wherein the evaluation unit is configured to prevent the initiation of the alarm routine when it determines that a temperature measured using the temperature sensor decreases by an amount exceeding a predefined threshold within a predetermined period of time.

7. The optical sensor device as defined in claim 5, wherein the blood perfusion parameter is indicative of a blood velocity in the tissue sample, wherein the sensor device further comprises an altimeter and wherein the evaluation unit is configured to determine the threshold on the basis of an altitude determined using the altimeter.

8. The optical sensor device as defined in claim 1, wherein the blood perfusion parameter is indicative of a blood velocity in the tissue sample, and wherein the sensor device further comprises a pressure sensor for measuring a pressure applied by the sensor device to the tissue sample, the evaluation unit being configured to detect changes of the pressure and to control the sensor device; to output a corresponding information when a change of the pressure is detected.

9. The optical sensor device as defined in claim 1, wherein the blood perfusion parameter is indicative of a blood velocity in the tissue sample, and wherein the sensor device further comprises a position sensor for detecting a displacement of the sensor device relative to the tissue sample, the evaluation unit being configured to control the sensor device to output a corresponding information when a displacement of the sensor device relative to the tissue sample is detected.

10. The optical sensor device; as defined in claim 1, wherein the evaluation unit is configured to determine plural values of the blood perfusion parameter according to different tissue depths on the basis of the contrast values determined for die distances.

11. The optical sensor device as defined in claim 10, wherein the evaluation unit is configured to determine the motion-corrected value of the blood perfusion parameter based on the values of the blood perfusion parameter according to the different tissue depths.

12. The optical sensor device as defined in claim 1, wherein the second opening is configured as an aperture, the size of the aperture being selected such that at least some speckles of the speckle pattern have a predetermined minimum size.

13. The optical sensor device as defined in claim 12, wherein a plurality of detection elements is associated with at least one distance to the light source and the light detection unit is configured to capture an image of the speckle pattern, the image comprising a plurality of pixels corresponding to the detection elements, and wherein the size of the aperture is selected such that a speckle having the minimum size covers at least two detection elements of the plurality of detection elements.

14. A method for determining at least one blood perfusion parameter of a user, the method comprising:
providing a sensor device comprising a light source for providing coherent light for scattering in a tissue sample of the user and a light detection unit for receiving at least part of the scattered coherent light, the light source and the light detection unit being arranged relative to each other in a re-emission geometry, and the light detection unit comprising plural light detection elements for capturing light intensity values in accordance with a speckle pattern formed by scattered coherent light, the light detection elements being arranged in increasing distances from the light source, where each distance is associated with a tissue depth corresponding to a tissue layer traversed by the light and where at least one light detection element is assigned to each distance;
bringing a contact surface of a housing of the sensor device into contact with the tissue sample, the contact surface comprising a first opening through which light emitted by the light source can leave the housing and a second opening through which scattered light captured by the light detection unit can enter the housing;
determining a contrast value for each distance based on light intensity captured by the at least one light detection element assigned to the respective distance, and
determining one motion-corrected value of the blood perfusion parameter based on the contrast values determined for the distances.
